(19) United States
(12) Patent Application Publication
(10) Pub. No.: US 2020/0315577 A1
(43) Pub. Date: Oct. 8, 2020

(54) ULTRASOUND PROBE

(71) Applicant: QISDA CORPORATION, Taoyuan City (TW)

(72) Inventor: Hsun-Pin Lee, Taoyuan City (TW)

(21) Appl. No.: 16/450,956

(22) Filed: Jun. 24, 2019

(30) Foreign Application Priority Data
Apr. 2, 2019 (TW) 108111763

(51) Int. Cl. A61B 8/00; B06B 1/06

(52) U.S. Cl. CPC A61B 8/4444; B06B 1/067; A61B 8/4488; B06B 2201/76; A61B 8/4494

(57) ABSTRACT

An ultrasound probe includes a transducer layer, a first matching layer, a lens layer, and a backing layer. The first matching layer is disposed at a first side of the transducer layer and a material of the first matching layer is PMMA or PO. The lens layer is disposed at the first side of the transducer layer and the first matching layer is located between the transducer layer and the lens layer. The backing layer is disposed at a second side of the transducer layer, wherein the first side is opposite to the second side.

### BACKGROUND OF THE INVENTION

### 1. Field of the Invention

The invention relates to an ultrasound probe and, more particularly, to an ultrasound probe capable of improving ultrasound image quality effectively.

### 2. Description of the Prior Art

Since ultrasound scanning equipment does not destroy material structure and cell, the ultrasound scanning equipment is in widespread use for the field of material and clinical diagnosis. In the prior art, the process of manufacturing an ultrasound probe comprises the following steps. First, an epoxy resin is coated and cured on a side of a piezoelectric material to form a matching layer. Then, a glue is poured and cured on another side of the piezoelectric material to form a backing layer. Then, a silicone is poured and cured on the epoxy resin to form a lens layer.

Referring to FIG. 1, FIG. 1 is a schematic diagram illustrating an ultrasound image scanned by an ultrasound probe of the prior art. As shown in FIG. 1, when the ultrasound probe of the prior art is performing ultrasound scanning, two opposite areas A1, A2 in the ultrasound image are obviously dark, such that an interpretation of the ultrasound image may be affected. Therefore, how to improve ultrasound image quality has become a significant design issue.

### SUMMARY OF THE INVENTION

An objective of the invention is to provide an ultrasound probe capable of improving ultrasound image quality effectively, so as to solve the aforesaid problem.

According to an embodiment of the invention, an ultrasound probe comprises a transducer layer, a first matching layer, a lens layer, and a backing layer. The first matching layer is disposed at a first side of the transducer layer and a material of the first matching layer is polymethyl methacrylate (PMMA) or polyolefin (PO). The lens layer is disposed at the first side of the transducer layer and the first matching layer is located between the transducer layer and the lens layer. The backing layer is disposed at a second side of the transducer layer, wherein the first side is opposite to the second side.

As mentioned in the above, the invention uses PMMA or PO to be the material of the first matching layer. When the ultrasound probe of the invention is performing ultrasound scanning, the number of characteristic points in the ultrasound image obviously increases. Accordingly, the invention can improve dark details in the ultrasound image, so as to improve ultrasound image quality effectively.

These and other objectives of the present invention will no doubt become obvious to those of ordinary skill in the art after reading the following detailed description of the preferred embodiment that is illustrated in the various figures and drawings.

### BRIEF DESCRIPTION OF THE DRAWINGS

FIG. 1 is a schematic diagram illustrating an ultrasound image scanned by an ultrasound probe of the prior art.

FIG. 2 is a schematic diagram illustrating an ultrasound probe according to an embodiment of the invention.

FIG. 3 is a schematic diagram illustrating an ultrasound image scanned by the ultrasound probe shown in FIG. 2.

FIG. 4 is a schematic diagram illustrating an ultrasound probe according to another embodiment of the invention.

### DETAILED DESCRIPTION

Referring to FIG. 2, FIG. 2 is a schematic diagram illustrating an ultrasound probe 1 according to an embodiment of the invention. As shown in FIG. 2, the ultrasound probe 1 comprises a transducer layer 10, a first matching layer 12, a second matching layer 14, a lens layer 16, and a backing layer 18. In this embodiment, the ultrasound probe 1 may be, but not limited to, a phased array ultrasound probe. The first matching layer 12, the second matching layer 14, and the lens layer 16 are disposed at a first side S1 of the transducer layer 10, and the backing layer 18 is disposed at a second side S2 of the transducer layer 10, wherein the first side S1 is opposite to the second side S2.

The invention may use a piezoelectric material to manufacture the transducer layer 10 first. Then, an epoxy resin is coated and cured on the first side S1 of the transducer layer 10 to form the second matching layer 14. In other words, a material of the second matching layer 14 is epoxy resin. Then, a glue is poured and cured on the second side S2 of the transducer layer 10 to form the backing layer 18. Then, a surface of the second matching layer 14 is cleaned. Then, polymethyl methacrylate (PMMA) or polyolefin (PO) is added onto the second matching layer 14 to form the first matching layer 12. In other words, a material of the first matching layer 12 is PMMA or PO. Then, a silicone is poured and cured on the first matching layer 12 to form the lens layer 16. After manufacturing the ultrasound probe 1, the first matching layer 12 and the second matching layer 14 are located between the transducer layer 10 and the lens layer 16, wherein the second matching layer 14 is located between the transducer layer 10 and the first matching layer 12.

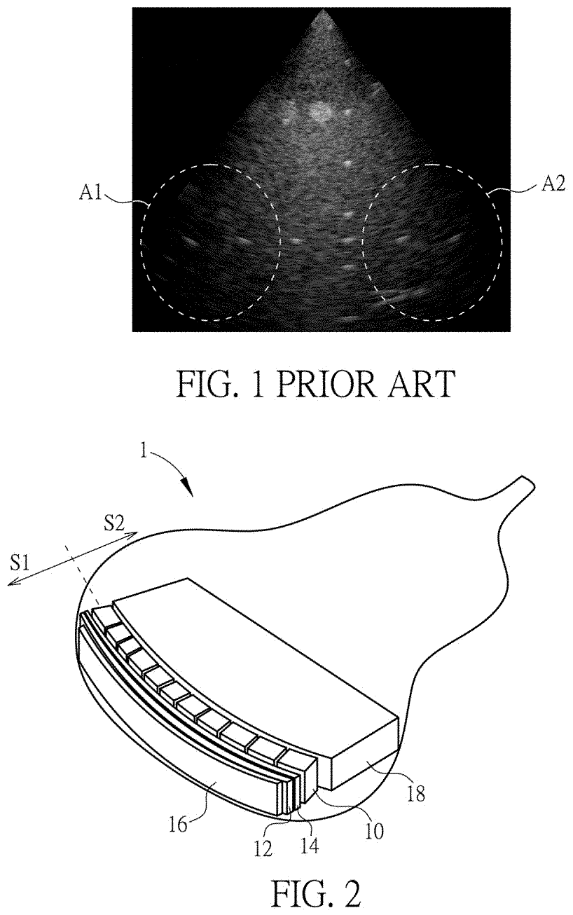

Referring to FIG. 3, FIG. 3 is a schematic diagram illustrating an ultrasound image scanned by the ultrasound probe 1 shown in FIG. 2. As shown in FIG. 3, when the ultrasound probe 1 of the invention is performing ultrasound scanning, the number of characteristic points of two opposite areas A1′, A2′ in the ultrasound image obviously increases. Compared to the two opposite areas A1, A2 in the ultrasound image of FIG. 1, the invention can improve dark details in the ultrasound image, so as to improve ultrasound image quality effectively.

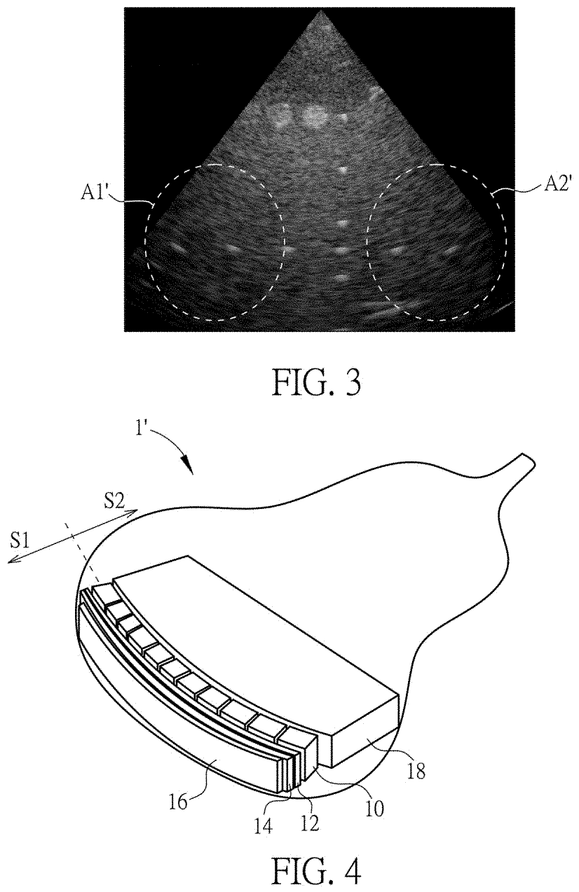

Referring to FIG. 4, FIG. 4 is a schematic diagram illustrating an ultrasound probe 1′ according to another embodiment of the invention. The main difference between the ultrasound probe 1′ and the aforesaid ultrasound probe 1 is that the second matching layer 14 of the ultrasound probe 1′ is located between the first matching layer 12 and the lens layer 16, as shown in FIG. 4. In other words, the first matching layer 12 of the ultrasound probe 1′ is located between the transducer layer 10 and the second matching layer 14.

It should be noted that the invention may only dispose the first matching layer 12 between the transducer layer 10 and the lens layer 16 without the second matching layer 14 according to practical applications.

As mentioned in the above, the invention uses PMMA or PO to be the material of the first matching layer and selectively uses epoxy resin to be the material of the second matching layer. When the ultrasound probe of the invention is performing ultrasound scanning, the number of characteristic points in the ultrasound image obviously increases. Accordingly, the invention can improve dark details in the ultrasound image, so as to improve ultrasound image quality effectively.

Those skilled in the art will readily observe that numerous modifications and alterations of the device and method may be made while retaining the teachings of the invention. Accordingly, the above disclosure should be construed as limited only by the metes and bounds of the appended claims.

What is claimed is:

1. An ultrasound probe comprising:
a transducer layer;
a first matching layer disposed at a first side of the transducer layer, a material of the first matching layer being polymethyl methacrylate or polyolefin;
a lens layer disposed at the first side of the transducer layer, the first matching layer being located between the transducer layer and the lens layer; and
a backing layer disposed at a second side of the transducer layer, the first side being opposite to the second side.

2. The ultrasound probe of claim 1, further comprising a second matching layer disposed at the first side of the transducer layer, the second matching layer being located between the transducer layer and the first matching layer.

3. The ultrasound probe of claim 2, wherein a material of the second matching layer is epoxy resin.

4. The ultrasound probe of claim 1, further comprising a second matching layer disposed at the first side of the transducer layer, the second matching layer being located between the first matching layer and the lens layer.

5. The ultrasound probe of claim 4, wherein a material of the second matching layer is epoxy resin.
